(19) United States
(12) Patent Application Publication
(10) Pub. No.: US 2026/0102093 A1
(43) Pub. Date: Apr. 16, 2026

(54) SYSTEMS AND METHODS FOR EMPLOYING A RECUPERATION MODE IN A VEHICLE

(71) Applicant: Toyota Jidosha Kabushiki Kaisha, Toyota-shi Aichi-ken (JP)

(72) Inventor: Tyler La Monda, Fairfield, CA (US)

(73) Assignee: Toyota Jidosha Kabushiki Kaisha, Toyota-shi Aichi-ken (JP)

(21) Appl. No.: 18/916,182

(22) Filed: Oct. 15, 2024

(51) Int. Cl. A61B 5/18

(52) U.S. Cl. CPC A61B 5/18

(57) ABSTRACT

A system includes one or more input devices of a vehicle configured to capture driver health state data pertaining to a health state of the driver, and one or more processors configured to compare the captured driver health state data to a threshold health state parameter, disable movement of the vehicle based on the comparison of the driver health state data to the threshold health state parameter, engage a recuperation mode in which one or more vehicle devices are configured to facilitate recuperation of the driver, and disengage the recuperation mode and enable movement of the vehicle based on an additional comparison of the driver health state data to the threshold health state parameter.

### FIELD

The present disclosure generally relates to systems and methods for employing a recuperation mode in a vehicle.

### BACKGROUND

Vehicle systems may seek to minimize or otherwise prevent distracted or impaired operation of a vehicle. However, conventional systems do not take any action to facilitate the recuperation of the driver to entertain the driver until the driver is fit to drive the vehicle and/or minimize the time the driver may be unfit to operate the vehicle.

### SUMMARY

In one embodiment, a system includes one or more input devices of a vehicle configured to capture driver health state data pertaining to a health state of the driver, and one or more processors configured to compare the captured driver health state data to a threshold health state parameter, disable movement of the vehicle based on the comparison of the driver health state data to the threshold health state parameter, engage a recuperation mode in which one or more vehicle devices are configured to facilitate recuperation of the driver, and disengage the recuperation mode and enable movement of the vehicle based on an additional comparison of the driver health state data to the threshold health state parameter.

In another embodiment, a method includes capturing driver health state data pertaining to a health state of a driver using one or more input devices positioned within a vehicle, comparing the captured driver health state data using one or more processors to a threshold health state parameter, disabling movement of the vehicle based on the comparison of the driver health state data to the threshold health state parameter, engaging a recuperation mode, with the one or more processors, in which one or more vehicle devices are configured to facilitate recuperation of the driver based on the comparison of the driver health state data to the threshold health state parameter, repeating capturing the driver health state data and comparing the captured driver health state data throughout a duration of the recuperation mode to monitor the health state of the driver, and disengaging the recuperation mode and enabling movement of the vehicle based on an additional comparison of the driver health state data to the threshold health state parameter.

In another embodiment, a vehicle includes one or more input devices configured to capture driver health state data pertaining to a health state of the driver, one or more processors, a memory module storing instructions that, when executed by the one or more processors, cause the vehicle to perform operations including capturing driver health state data pertaining to a health state of a driver using the one or more input devices, comparing the captured driver health state data to a threshold health state parameter, disabling movement of the vehicle based on the comparison of the driver health state data to the threshold health state parameter, engaging a recuperation mode in which one or more vehicle devices are configured to facilitate recuperation of the driver based on the comparison of the driver health state data to the threshold health state parameter, repeating capturing the driver health state data and comparing the captured driver health state data throughout a duration of the recuperation mode to monitor the health state of the driver, and disengaging the recuperation mode and enabling movement of the vehicle based on an additional comparison of the driver health state data to the threshold health state parameter.

These and other features, and characteristics of the present technology, as well as the methods of operation and functions of the related elements of structure and the combination of parts and economies of manufacture, will become more apparent upon consideration of the following description and the appended claims with reference to the accompanying drawings, all of which form a part of this specification, wherein like reference numerals designate corresponding parts in the various figures. It is to be expressly understood, however, that the drawings are for the purpose of illustration and description only and are not intended as a definition of the limits of the disclosure. As used in the specification and in the claims, the singular form of ‘a’, ‘an’, and ‘the’ include plural referents unless the context clearly dictates otherwise.

### BRIEF DESCRIPTION OF THE DRAWINGS

The embodiments set forth in the drawings are illustrative and exemplary in nature and not intended to limit the subject matter defined by the claims. The following detailed description of the illustrative embodiments can be understood when read in conjunction with the following drawings, wherein like structure is indicated with like reference numerals and in which:

FIG. 1 depicts a schematic diagram of an example system for employing a recuperation mode in a vehicle, according to one or more embodiments shown and described herein;

FIG. 2 depicts a schematic diagram of an example system for capturing driver health state data and providing a recuperation mode, according to one or more embodiments shown and described herein; and

FIG. 3 depicts a flow diagram of an example method to be performed by a processor, according to one or more embodiments shown and described herein.

### DETAILED DESCRIPTION

Conventional systems that may restrict driving of a vehicle are limited for two primary reasons. First, these systems typically do not assess a range of driver heath state data to determine a more complete understanding of the driver's health state prior to disabling access to the vehicle for an unspecified amount of time. Second, conventional systems to prevent impaired driving do not provide resources to help facilitate the recuperation of the driver to entertain the driver until the driver is fit to the driver the vehicle and/or limit the amount of time the vehicle must be rendered inoperable.

Accordingly, there is a need for systems and methods for capturing driver health state data pertaining to a health state of the driver, restricting operation of a vehicle based on a comparison with stored threshold health state parameters, and providing a recuperation mode featuring one or more recuperation strategies implemented by vehicle devices positioned within the passenger compartment of the vehicle. In embodiments, the present disclosure provides a system and method for automatically detecting a heath state of a driver, restricting the operation of the vehicle if the driver is deemed to be unfit to drive, and providing a recuperation procedure to entertain the driver until the driver is fit to the driver the vehicle and/or help to reduce the time the driver remains unfit to operate the vehicle. As such, the present disclosure provides for systems and methods that can adaptively determine a health state of the driver to prevent impaired driving, while also providing both active and passive recuperation techniques that may entertain a driver until the driver is fit to drive and/or to expedite a driver's recuperation time.

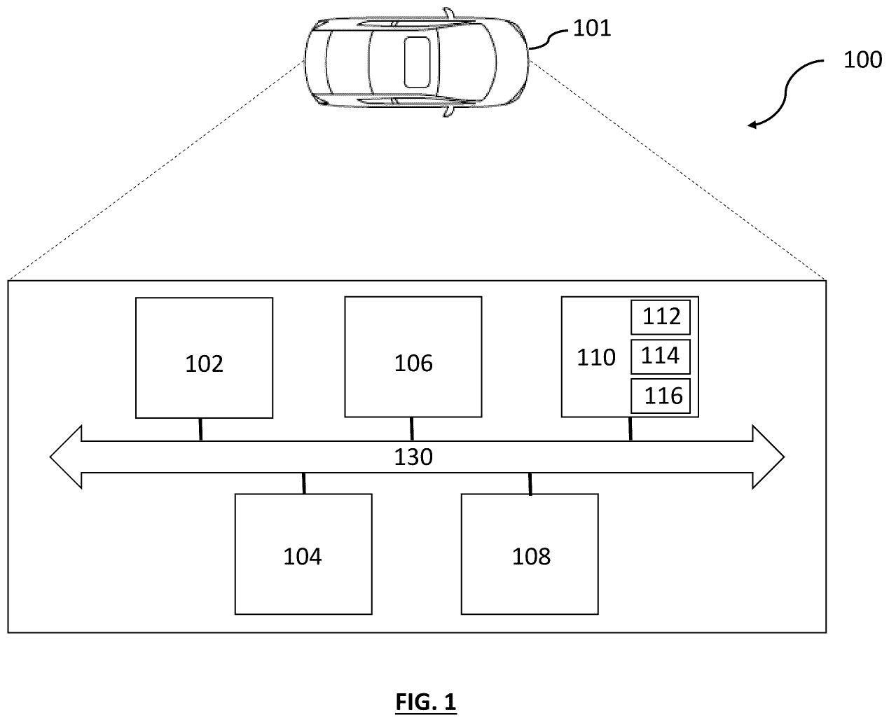

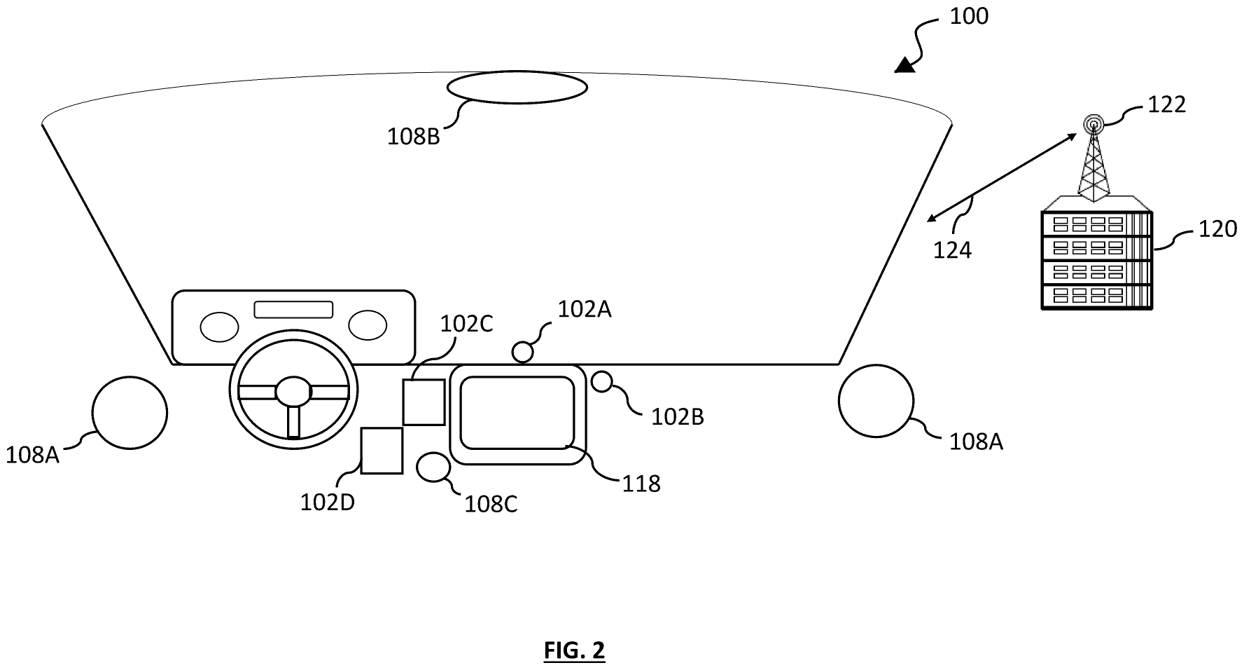

FIGS. 1 and 2 depict schematic diagrams of a system 100. The system 100 includes a vehicle 101, at least one processor 106, one or more input devices 102, a memory module 110, and one or more vehicle devices 108. In some embodiments, the system 100 additionally includes one or more communication devices 104 and one or more servers 120 in communication with the processor 106 via the one or more communication devices 104. Although FIG. 1 illustrates single instances of the constituent components of the system 100, the system 100 may include any number of constituent components.

As depicted in FIG. 1, the system 100 includes a vehicle 101. The vehicle 101 may be any one of a passenger vehicle, a non-passenger vehicle, a taxi, a bus, a scooter, a motorcycle, a truck, or any other type of motorized or electric vehicle. The vehicle 101 may move or appear on various surfaces, such as, without limitations, roads, highways, streets, expressways, bridges, tunnels, parking lots, garages, off-road trails, railroads, or any surfaces where the vehicles may operate. Additionally, the vehicle 101 may include a GPS unit that is capable of determining a past, present, and/or future spatial position of the vehicle 101. The GPS unit may receive and communicate relevant positional information related to the vehicle such as latitude and longitude coordinates, standard GPS coordinates, a GPS trace, or a partial GPS trace.

Still referring to FIG. 1, as described above, the system 100 includes one or more processors 106 positioned within the vehicle 101 and placed in communication with various components, including the one or more vehicle input devices 102, the memory module 110, the one or more communication devices 104, and the one or more vehicle devices 108 via a communication path 130 that provides signal interconnectivity between various components of the system 100. Accordingly, the communication path 130 may communicatively couple any number of processors 106 with one another, and allow the components coupled to the communication path 130 to operate in a distributed computing environment. Specifically, each of the components may operate as a node that may send and/or receive data. As used herein, the term “communicatively coupled” means that coupled components are capable of exchanging data signals with one another such as, for example, electrical signals via a conductive medium, electromagnetic signals via air, optical signals via optical waveguides, and the like.

Accordingly, the communication path 130 may be formed from any medium that is capable of transmitting a signal such as, for example, conductive wires, conductive traces, optical waveguides, or the like. In some embodiments, the communication path 130 may facilitate the transmission of wireless signals, such as WiFi, Bluetooth®, Near Field Communication (NFC), and the like. Moreover, the communication path 130 may be formed from a combination of mediums capable of transmitting signals. Accordingly, the communication path 130 may comprise a vehicle bus, such as for example a LIN bus, a CAN bus, a VAN bus, and the like. Additionally, it is noted that the term “signal” means a waveform (e.g., electrical, optical, magnetic, mechanical, or electromagnetic), such as DC, AC, sinusoidal wave, triangular wave, square-wave, vibration, and the like, capable of traveling through a medium.

The processor 106 may be a controller, an integrated circuit, a microchip, a computer, or any other computing device. The processor 106 may be configured to perform operations, make calculations or execute one or more executable programs stored on the memory module 110. The processor 106 may be any suitable device known to those of ordinary skill in the art such as a processing device, computing device, or combinations thereof, including, for example, a multi-core processor, a microcontroller, a field-programmable gate array (FPGA), or an application-specific integrated circuit (ASIC). The processor 106 may also include further auxiliary processing components configured to receive and execute operations, instructions, or programs. In embodiments, the processor 106 may execute one or more software applications that enable remote communications with one or more components of the system 100. In embodiments, the system 100 may include one or more communication devices 104 such has as transceiver to place the processor 106 in communication with a remote server 120.

The memory module 110 is communicatively coupled to the communication path 130. The memory module 110 may comprise RAM, ROM, flash memories, hard drives, or any device capable of storing machine-readable and executable instructions such that the machine-readable and executable instructions can be accessed by the one or more processors 106. The machine-readable and executable instructions may comprise one or more logic or algorithms written in any programming language of any generation (e.g., 1GL, 2GL, 3GL, 4GL, or 5GL) such as, for example, machine language that may be directly executed by the processor 106, or assembly language, object-oriented programming (OOP), scripting languages, microcode, etc., that may be compiled or assembled into machine-readable and executable instructions and stored on the memory module 110. Alternatively, the machine-readable and executable instructions may be written in a hardware description language (HDL), such as logic implemented via either a field-programmable gate array (FPGA) configuration or an application-specific integrated circuit (ASIC), or their equivalents. Accordingly, the methods described herein may be implemented in any conventional computer programming language, as pre-programmed hardware elements, or as a combination of hardware and software components. The one or more processor 106 along with the memory module 110 may operate as a controller or an electronic control unit (ECU) for the vehicle 101 or any other components of the system 100. In some embodiments, the memory module 110 may comprise two or more memory devices communicatively coupled along the communication path 130.

Referring collectively to FIGS. 1 and 2, the system 100 also includes one or more input devices 102 positioned within or about the vehicle 101 and placed in communication with the processor 106 along the communication path 130. The vehicle input devices 102 are configured to capture driver health state data pertaining to a health state of a driver. The one or more vehicle input devices 102 may be positioned about the passenger compartment of the vehicle 101 to directly measure and collect the driver health state data and communicate the collected driver health state data to the processor 106. In embodiments, the one or more vehicle input devices 102 may be positioned centrally or substantially spaced about the passenger compartment of the vehicle 101 to collect driver health state data throughout an entirety of the passenger compartment of the vehicle 101. In other embodiments, the one or more vehicle input devices 102 may be positioned proximate to a driver's seat or may be otherwise positioned to directly capture the driver health state data while a driver is seated in the driver's seat. In some embodiments, the vehicle input devices 102 may be positioned on an exterior of the vehicle 101 to capture driver health state data pertaining to a health state of a driver as the driver approaches the vehicle 101.

The driver health state data may comprise photographic or videographic data capturing the physical movements of the driver within or near the vehicle 101. In embodiments, the driver health state data comprises photographic or videographic data of a gait of the driver, a posture of the driver, hand or arm movements of the driver, eye movements of the driver, head movements of the driver, or other comparable physical characteristics that may indicate a health state of the driver. In embodiments, the driver health state data comprises audio data pertaining to the speech patterns of the driver that may indicate a health state of the driver. In embodiments, the driver health state data comprises gas data or odor data pertaining to the body odor or the perspiration of the driver that may indicate the driver's health state. The driver health state data may also comprise collected biological data including data pertaining to a collected breath sample of the driver, a collected saliva sample of the driver, or a collected blood sample of the driver. In embodiments, the driver health state data may include a single type or category of driver health state data discussed above. In other embodiments, the one or more input devices 102 may collect a combination of the various types of driver health state data discussed herein.

The one or more input devices 102 may include cameras 102A. The cameras 102A may be conventional optical cameras, infrared cameras, LIDAR sensors, or any other type of imaging sensor capable of capturing photographic or videographic data pertaining to the health state of the driver. The cameras 102A may be conventional cameras 102A of the vehicle 101 that are being used for a secondary purpose or the cameras 102A may be dedicated cameras 102A configured exclusively to collect the driver health state data. The one or more input devices 102 may include microphones 102B. The microphones 102B may be any transducer capable of converting sound into an electrical signal. The microphones 102B may be conventional microphones 102B of the vehicle 101 that are being used for a secondary purpose or the microphones 102B may be dedicated microphones 102B configured exclusively to collect the driver health state data. The one or more input devices 102 may include aroma sensors 102C such as a gas sensor capable of detecting and identifying odors within the interior of the passenger compartment of the vehicle 101. In embodiments, the aroma sensor 102C may be an electronic nose comprising a chemical sensor array to detect the presence of particular chemical compounds on a sensor surface. Furthermore, the one or more input devices 102 may include a breathalyzer 102D or alcohol meter. In some embodiments, the one or more input devices 102 may include one or more additional biological testing devices such as a blood glucose monitoring device, a saliva testing device, or any other comparable portable testing instrument configured to collect driver health state data pertaining to a health state of the driver.

In embodiments, the plurality of input devices 102 may include a single type or category of vehicle input device 102 discussed above. In other embodiments, the one or more input devices 102 may include a combination of the various types of input devices 102 discussed above. For example, in exemplary systems, the one or more input devices 102 may include any number of internal cameras 102A, microphones 102B, aroma sensors 102C, or breathalyzers 102D, each communicating a respective driver health state data input to the processor 106. The one or more input devices 102 may be integrally formed with the vehicle 101 such that the input devices cannot be removed. Alternatively, the one or more input devices 102 may be remote devices or portable devices that may collect and transmit driver health state data to the processor 106 remotely.

Still referring to FIGS. 1 and 2, the vehicle 101 may include one or more communication devices 104 operable to wirelessly communicate with one or more external servers 120. The server 120 may include corresponding communication hardware 122 to enable the transmission of a wireless data transfer 124 between the vehicle 101 and the one or more servers 120. In embodiments, the one or more servers 120 may include, without limitation, one or more of cloud servers, smartphones, tablets, telematics servers, fleet management servers, connected car platforms, application servers, Internet of Things (IoTs) servers, or any server with the capability to transmit data with vehicles. The wireless data transfer 124 may include the use of one or more computer networks (e.g., a personal area network, a local area network, or a wide area network), cellular networks, satellite networks and/or a global positioning system and combinations thereof. Accordingly, the vehicles 101 and the servers 120 can wirelessly transfer data via a wide area network, via a local area network, via a personal area network, via a cellular network, via a satellite network, etc. Suitable local area networks may include wired Ethernet and/or wireless technologies such as, for example, Wi-Fi. Suitable personal area networks may include wireless technologies such as, for example, IrDA, Bluetooth®, Wireless USB, Z-Wave, ZigBee, and/or other near-field communication protocols. Suitable cellular networks include, but are not limited to, technologies such as LTE, WiMAX, UMTS, CDMA, and GSM.

Still referring to FIGS. 1 and 2, in embodiments where the system 100 comprises the communication device 104 and one or more server 120, the system 100 can transmit health information of the driver from the server 120. In embodiments, the health information may be a communicated health profile corresponding to the physical characteristics and/or one or more health diagnoses of the driver. In embodiments, the health profile of the driver may be transmitted from a secure database of medical records, authorized in advance by the driver. The health profile may, for instance, contain physical information of the driver including the driver's height, weight, age, and other demographic information. The health profile may contain past diagnoses or medical information including, but not limited to, past treatment for epilepsy, cardiac dysrhythmia, dementia, visual disabilities, anxiety attacks, and substance dependency. The processor 106 and/or the server 120 may, alone or in combination, evaluate the captured driver health state data in conjunction with the communicated health profile to assess the health state of the driver.

The processor 106 may be configured to compare the captured driver health state data against a threshold health state parameter 116. The processor 106 may receive and aggregate driver health state data from the one or more input devices 102 to determine a health state of the driver by determining whether the measured driver health state data does not lie within an acceptable range as defined by the threshold health state parameter 116. If the comparison performed by the processor 106 determines that the measured driver health state data does not meet the threshold health state parameter 116, the processor 106 will categorize the driver's health state as being unfit to drive the vehicle 101 and subsequently disable operation of the vehicle 101. The processor 106 may perform the collection, aggregation, and comparison of the measured driver health state data locally. In embodiments, the processor 106 may transmit some or all of the driver health state data via the communication device 104 to perform one or more of the collection, aggregation, and comparison of the measured driver health state data in a cloud computing environment on the server 120. In embodiments, the processor 106 can prevent the vehicle 101 from starting if the processor 106 determines that the measured driver health state data does not meet the threshold health state parameter 116. Additionally, the processor 106 can notify a driver to stop the vehicle 101 during operation and disable the vehicle 101 once stationary if the processor 106 determines that the measured driver health state data does not meet the threshold health state parameter 116 during the operation of the vehicle 101. In embodiments, the system 100 may further comprise a vehicle shutoff device, such as an electric or manual shutoff device, in communication with the processor 106 to enable the processor to indirectly disable operation of the vehicle 101 if the processor 106 determines that the measured driver health state data does not meet the threshold health state parameter 116.

The threshold health state parameter 116 may be one or more sets of data stored in any one of the memory module 110, the server 120, or on a local or remote database that represent a threshold value corresponding to a particular type of driver health state data collected by the one or more input devices 102. For instance, in embodiments where the driver health state data comprises gas data or odor data pertaining to the body odor or the perspiration of the driver, the threshold health state parameter 116 may comprise a threshold value for a particular gas or odor present in the air. In an embodiment, the threshold health state parameter 116 may be a data value corresponding to an allowable amount of alcohol vapor in the air inside the vehicle 101. In embodiments, the threshold health state parameter 116 may be a particular concentration of alcohol vapor within the passenger compartment of the vehicle 101, above which the processor 106 will disable operation of the vehicle 101. In some embodiments, the threshold health state parameter 116 may be a binary value representing the presence or absence of alcohol vapor within the passenger compartment of the vehicle 101. In embodiments where driver health state data comprises collected biological data, the threshold health state parameter 116 may be a threshold quantitative value of a particular characteristic within the biological sample that indicates that the health state of the driver is not fit to operate the vehicle 101. For instance, in embodiments where the driver health state data comprises a collected breath or blood sample, the threshold health state parameter 116 may comprise a threshold blood alcohol content (BAC), above which the processor 106 will disable operation of the vehicle 101. In embodiments, the threshold health state parameter 116 may be a BAC of 0.08 g/100 ml. The processor 106 may compare a single type of measured driver health state data against the threshold health state parameter 116 or the processor 106 may compare multiple types of measured driver health state data against one or more respective threshold health state parameters 116 to determine whether the health state of the driver is fit for operation of the vehicle 101. In embodiments, the processor 106 may disable the operation of the vehicle 101 if any measured driver health state data does not lie within an acceptable range as defined by the corresponding threshold health state parameter 116. In some embodiments, the processor 106 may compare multiple types of driver health state data against their respective threshold health state parameters 116 to corroborate and validate the measured driver health state data prior to disabling the operation of the vehicle 101.

In embodiments, the memory module 110 may further comprise a context builder 112 to determine and store a baseline health state condition and compare the driver health state data against the baseline health state condition and/or threshold health state parameter 116. The context builder 112 may include instructions that, when executed by the processor 106, aggregate and process photographic, videographic, or audio driver health state data over a period of time pertaining to the gait of the driver, the posture of the driver, the hand or arm movements of the driver, the eye movements of the driver, the head movements of the driver, speech patterns, speech volumes, or other comparable physical characteristics. Based on the aggregation of this driver health state data, the context builder 112 may include instructions that, when executed by the processor 106, establish a baseline health state condition comprising a profile of normal or baseline physical characteristics. For instance, the baseline health state condition may comprise a baseline profile further comprising measurements of a standard walking posture of the driver, measurements of the standard sitting posture of the driver, measurements of the standard arm or hand movements of the driver, measurements of the standard eye movements of the driver, measurements of the standard head movements of the driver, measurements of speaking speed, measurements of speaking fluidity, and measurements of speaking volume. Additionally, the context builder 112 may include instructions that, when executed by the processor 106, evaluate the photographic, videographic, or audio driver health state data to determine and/or adjust one or more threshold health state parameters 116. For instance, the context builder 112 may include instructions that, when executed by the processor 106, determine a threshold walking speed, a threshold number of stumbles or walking irregularities, a threshold number of eye movements per second, a threshold speaking volume, a threshold number of speech irregularities, or other comparable movement or speech metrics to be stored as a threshold health state parameter 116. The processor 106 may compare a single type of measured driver health state data against the threshold health state parameter 116 or the baseline health state condition. Additionally, the processor 106 may compare multiple types of measured driver health state data against one or more respective threshold health state parameters 116 or the baseline health state condition to determine whether the health state of the driver is fit for operation of the vehicle 101. In embodiments, the processor 106 may disable the operation of the vehicle 101 if any measured driver health state data does not lie within an acceptable range as defined by the corresponding threshold health state parameter 116 or deviates a threshold percentage from the measurements stored in the baseline health state condition. For instance, the processor 106 may disable the operation of the vehicle 101 if the driver health state data deviates by 5%, 10%, 25%, or even 50% from the standard measurements stored in the baseline health state condition. In some embodiments, the processor 106 may compare multiple types of driver health state data against their respective threshold health state parameters 116 and/or baseline health state condition to corroborate and validate the measured driver health state data prior to disabling the operation of the vehicle 101.

In embodiments, the memory module 110 further comprises a prediction module 114 to predict a recuperation time representing a length of time before the measured driver health state data predicted to be within an acceptable range as defined by the one or more threshold health state parameters 116. In embodiments, the prediction module 114 may include instructions that, when executed by the processor 106, further predict the recuperation time by comparing the driver health state data against a health profile of the driver. The processor 106 may evaluate the magnitude by which the measured driver health state data does not lie within an acceptable range as defined by the one or more threshold health state parameters 116 in context with one or more physical characteristics of the driver contained in the health profile to calculate a personalized prediction for when the driver will once again be fit to operate the vehicle 101. The health profile of the driver may comprise a plurality of health information data personalized to the physical characteristics of the particular driver. For instance, the prediction module 114 may contain instructions that, when executed by the processor 106, consider the age, height, and weight of a driver to predict a more accurate recuperation time when a BAC of the driver is determined to exceed or otherwise not lie within an acceptable range as defined by the threshold health state parameter 116 corresponding to the BAC of the driver. The prediction module 114 may contain instructions that, when executed by the processor 106, generate a predicted recuperation time estimate to be displayed to the driver on a user interface 118. In embodiments, if the predicted recuperation time calculated by the instructions of the prediction module 114 exceeds a threshold time value, the one or more communication devices 104 may communicate with the external server 120 or another third-party device to solicit a ride for the driver. In embodiments, the threshold time value may be greater than or equal to 1 hour, greater than equal to 2 hours, greater than or equal to 4 hours, or even greater than or equal to 6 hours.

The health profile of the driver may be stored locally within the memory module 110 of the vehicle 101 or the health profile of the driver may be stored remotely and transmitted from the server 120 to the processor 106. In embodiments, the health profile of the driver comprises the baseline health state condition calculated by the instructions included in the context builder 112. In embodiments, the health profile of the driver comprises one or more threshold health state parameters 116. In embodiments, the health profile of the driver contain physical information of the driver including the driver's height, weight, age, and other demographic information. The health profile may contain past diagnoses or medical information including, but not limited to, past treatment for epilepsy, cardiac dysrhythmia, dementia, visual disabilities, anxiety attacks, and substance dependency. In embodiments, the processor 106 may evaluate the heath profile of the driver and adjust the values of the one or more threshold health state parameters 116 accordingly. For instance, if the health profile of the driver contains a past medical diagnosis for epilepsy, the processor 106 may accordingly adjust the threshold health state parameters 116 corresponding to head movement and eye movement variations to more quickly disable operation of the vehicle 101 for a driver with a documented history of experiencing seizures.

Once the processor 106 has disabled the operation of the vehicle 101 due to the measured driver health state data does not lie within an acceptable range as defined by the one or more threshold health state parameters 116, the processor 106 may engage a recuperation mode to facilitate recuperation of the driver. During the recuperation mode, the processor 106 may engage one or more vehicle devices 108 positioned within the passenger compartment of the vehicle 101 to facilitate recuperation of the driver. In embodiments, the one or more vehicle devices 108 may comprise audio speakers 108A positioned around the passenger compartment of the vehicle 101 and configured to play audio recordings. In embodiments, the one or more vehicle devices 108 may comprise LED lights 108B positioned around the passenger compartment of the vehicle 101 and configured to illuminate the passenger compartment of the vehicle 101 with selected colors and light intensities. In embodiments, the one or more vehicle devices 108 may comprise aroma dispensers 108C positioned within the passenger compartment of the vehicle 101 and configured to dispense a controlled amount of a stored smelling fluid to selectively control the aroma or odor within the vehicle 101. In embodiments, the one or more vehicle devices 108 may comprise the user interface 118. In embodiments, each of the one or more vehicle devices 108 may be directly controlled by processor 106. The processor 106 may engage a standard procedure to operate one or more vehicle devices 108 or the processor 106 may engage a personalized procedure to selectively operate one or more vehicle devices 108 according to a programmed user preference or according to the health profile of the driver.

The one or more vehicle devices 108 may be configured to facilitate recuperation the health state of the driver by providing passive treatments to calm the driver and provide a restful environment to recuperate without the need for direct action from the driver. In embodiments, one or more aroma dispensers 108C may be positioned proximate the driver to provide aroma therapy by selectively releasing a controlled amount of a scented liquid. In embodiments, one or more audio speakers 108A may be positioned within the passenger compartment of the vehicle 101 to play audio recordings at a controlled volume to calm the driver. In embodiments, one or more lights 108B may be positioned within the passenger compartment of the vehicle 101 to selectively control the lighting, including varying the light color and varying the light brightness, within the passenger compartment of the vehicle 101. In embodiments, the lights 108B may also be configured to project calming visuals on a surface within the vehicle 101. In embodiments, the user interface 118 may be configured to provide calming visuals for the driver. The passive treatments engaged by the processor 106 may remain constant throughout the recuperation mode or may be dynamically variable throughout the duration of the recuperation mode to facilitate recuperation the evolving health state of the driver. In embodiments, the driver may selectively engage or disengage one or more of the vehicle devices 108 via the user interface 118.

The one or more vehicle devices 108 may be configured to facilitate recuperation of the driver by providing active treatments to engage the driver and aid in recuperation due to the direct actions of the driver. In embodiments, the one or more vehicle devices 108 may be configured to facilitate recuperation of the driver by providing one or more activities on the user interface 118 of the vehicle 101 to solicit the driver's direct engagement. The activities may include games, puzzles, and/or tasks displayed on a user interface 118 that are configured to require the driver to interact with the user interface 118 to complete the activity. In embodiments, the processor 106 may grade the driver's performance in completing the activity on the user interface 118 and assign the driver a performance metric based on the accuracy and/or efficiency in completing the activity. In embodiments, the processor 106 may evaluate the performance metric in addition to one or more of the threshold health state parameter 116, the baseline health state condition, and the health profile of the driver when determining when to disengage the recuperation mode and enable movement of the vehicle 101.

During the recuperation mode, the processor 106 may direct the one or more input devices 102 to repeatedly capture driver health state data and the processor 106 may continuously compare the additional captured driver health state data throughout the duration of the recuperation mode to continuously monitor the health state of the driver. In some embodiments, the processor 106 may direct the one or more input devices 102 to capture additional driver health state data at predetermined check points throughout the duration of the recuperation mode, rather than capturing driver health state data continuously. The predetermined check points may be time intervals. In embodiments, the checkpoints may be every 15 minutes, every 30 minutes, or even every hour. In other embodiments, the predetermined check points may be associated with the completion of an activity on the user interface 118. The checkpoint may comprise requiring the driver to achieve a required performance metric on an activity displayed on the user interface 118. One the activity is completed, the processor 106 may direct the one or more input devices 102 to capture additional driver health state data and compare the captured driver health state data against one or more threshold health state parameters 116. Once the captured driver health state data meets the threshold health state parameter 116, the processor may disengage the recuperation mode and enable movement of the vehicle 101. In embodiments, the processor 106 may perform additional validation comparisons to ensure that the driver is healthy enough to drive prior to enabling movement of the vehicle 101.

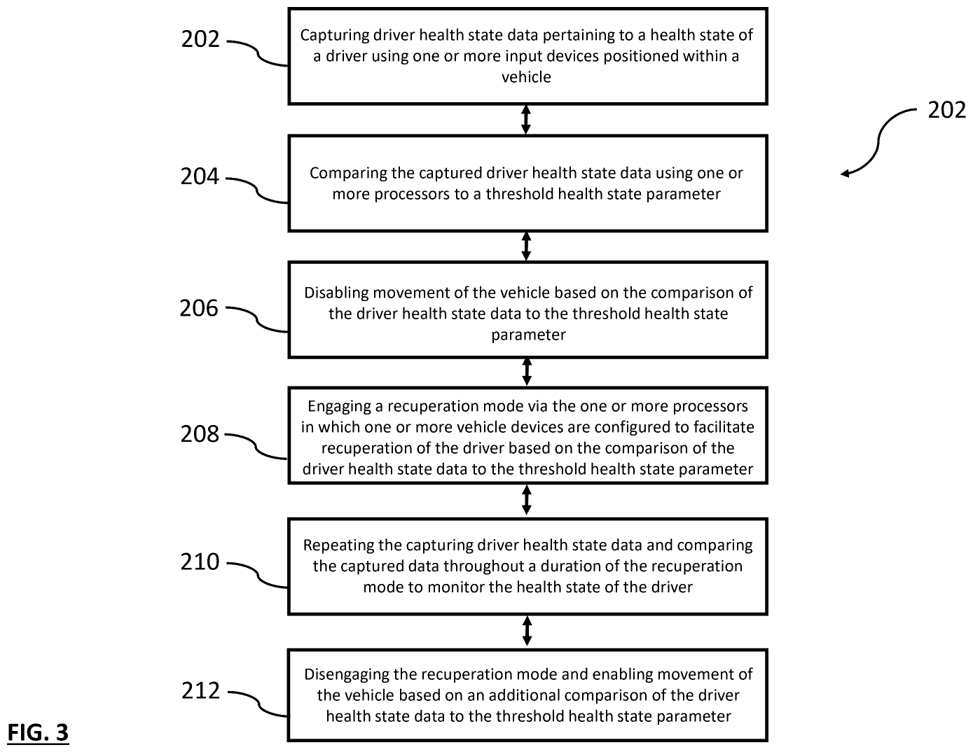

FIG. 3 depicts a flow diagram of an example method 200 performed by the processor 106. FIG. 3 may reference and incorporate any of the above constituent components and corresponding disclosure explained above with respect to FIGS. 1 and 2, such as the example system 100.

At block 202, the system 100 may capture driver health state data pertaining to a health state of a driver using one or more input devices positioned within a vehicle. In embodiments, the one or more input devices further comprise one or more of internal cameras, microphones, aroma sensors, or a breathalyzer positioned within the passenger cab of the vehicle 101.

At block 204, one or more processor 106 may be configured to compare the captured driver health state data against a threshold health state parameter 116. In embodiments, the method 200 further comprises determining a baseline health state condition using a context builder and comparing the captured driver health state data against the baseline health state condition.

At block 206, based on the comparison, the processor 106 is configured to disable movement of the vehicle 101 if the driver health state data does not lie within an acceptable range as defined by the threshold health state parameter 116. In embodiments, the processor 106 may receive and aggregate driver health state data from the one or more input devices 102 to determine a health state of the driver by determining whether the measured driver health state data does not lie within an acceptable range as defined by the threshold health state parameter 116. If the comparison performed by the processor 106 determines that the measured driver health state data does not meet the threshold health state parameter 116, the processor 106 will categorize the driver's health state as being unfit to drive the vehicle 101 and subsequently disable operation of the vehicle 101.

At block 208, the processor 106 is configured to engage a recuperation mode via the one or more processors wherein one or more vehicle devices are configured to facilitate recuperation of the driver based on the comparison of the driver health state data to the threshold health state parameter, such as if the driver health state data does not lie within an acceptable range as defined by the threshold health state parameter. In embodiments, the system further comprises predicting a recuperation time using a prediction module to determine a duration of the recuperation mode. Moreover, in embodiments, the one or more vehicle devices may be configured to facilitate recuperation of the driver by providing passive treatments including one or more of aroma therapy, calming audio, and calming visuals. In other embodiments, the one or more vehicle devices may be configured to facilitate recuperation of the driver by providing an activity including one or more of games, puzzles, and tasks on a user interface.

At block 210, the processor 106 is configured to repeat the capturing driver health state data and comparing the captured data throughout a duration of the recuperation mode to monitor the health state of the driver. In embodiments, the one or more input devices may continuously capture driver health state data pertaining to the health state of the driver. In other embodiments, the one or more input devices may selectively capture driver health state data pertaining to the health state of the driver at pre-determined check points to maximize processing efficiency and reduce unnecessary data storage.

At block 212, the method 200 includes disengaging the recuperation mode and enabling movement of the vehicle when the driver health state data based on an additional comparison of the driver health state data to the threshold health state parameter, such as when the driver health state data meets the threshold health state parameter. In embodiments, disengaging the recuperation mode and enabling movement of the vehicle further comprises evaluating a performance metric of the activity.

Accordingly, embodiments of the present disclosure satisfy the need for systems and methods for capturing driver health state data pertaining to a health state of the driver, restricting operation of a vehicle based on a comparison with stored threshold health state parameters, and providing a recuperation mode featuring one or more recuperation strategies implemented by vehicle devices positioned within the passenger compartment of the vehicle. In embodiments, the present disclosure provides a system and method for automatically detecting a heath state of a driver, restricting the operation of the vehicle if the driver is deemed to be unfit to drive, and providing a recuperation procedure to entertain the driver until the driver is fit to the driver the vehicle and/or help to reduce the time the driver remains unfit to operate the vehicle. As such, the present disclosure provides for systems and methods that can adaptively determine a health state of the driver to prevent impaired driving, while also providing both active and passive recuperation techniques that may entertain a driver until the driver is fit to drive and/or to expedite a driver's recuperation time.

The preceding description is provided to enable any person skilled in the art to practice the various embodiments described herein. The examples discussed herein are not limiting of the scope, applicability, or embodiments set forth in the claims. Various modifications to these embodiments will be readily apparent to those skilled in the art, and the generic principles defined herein may be applied to other embodiments. For example, changes may be made in the function and arrangement of elements discussed without departing from the scope of the disclosure. Various examples may omit, substitute, or add various procedures or components as appropriate. For instance, the methods described may be performed in an order different from that described, and various steps may be added, omitted, or combined. Also, features described with respect to some aspects may be combined in some other aspects. For example, an apparatus may be implemented or a method may be practiced using any number of the aspects set forth herein. In addition, the scope of the disclosure is intended to cover such an apparatus or method that is practiced using other structure, functionality, or structure and functionality in addition to, or other than, the various aspects of the disclosure set forth herein. It should be understood that any aspect of the disclosure disclosed herein may be embodied by one or more elements of a claim.

As used herein, the word “exemplary” means “serving as an example, instance, or illustration.” Any aspect described herein as “exemplary” is not necessarily to be construed as preferred or advantageous over other aspects.

As used herein, a phrase referring to “at least one of” a list of items refers to any combination of those items, including single members. As an example, “at least one of: a, b, or c” is intended to cover a, b, c, a-b, a-c, b-c, and a-b-c, as well as any combination with multiples of the same element (e.g., a-a, a-a-a, a-a-b, a-a-c, a-b-b, a-c-c, b-b, b-b-b, b-b-c, c-c, and c-c-c or any other ordering of a, b, and c). Reference to an element in the singular is not intended to mean only one unless specifically so stated, but rather “one or more.” For example, reference to an element (e.g., “a processor,” “a memory,” etc.), unless otherwise specifically stated, should be understood to refer to one or more elements (e.g., “one or more processors,” “one or more memories,” etc.). The terms “set” and “group” are intended to include one or more elements, and may be used interchangeably with “one or more.” Where reference is made to one or more elements performing functions (e.g., steps of a method), one element may perform all functions, or more than one element may collectively perform the functions. When more than one element collectively performs the functions, each function need not be performed by each of those elements (e.g., different functions may be performed by different elements) and/or each function need not be performed in whole by only one element (e.g., different elements may perform different sub-functions of a function). Similarly, where reference is made to one or more elements configured to cause another element (e.g., an apparatus) to perform functions, one element may be configured to cause the other element to perform all functions, or more than one element may collectively be configured to cause the other element to perform the functions. Unless specifically stated otherwise, the term “some” refers to one or more.

As used herein, the term “determining” encompasses a wide variety of actions. For example, “determining” may include calculating, computing, processing, deriving, investigating, looking up (e.g., looking up in a table, a database or another data structure), ascertaining and the like. Also, “determining” may include receiving (e.g., receiving information), accessing (e.g., accessing data in a memory) and the like. Also, “determining” may include resolving, selecting, choosing, establishing and the like.

The methods disclosed herein include one or more steps or actions for achieving the methods. The method steps and/or actions may be interchanged with one another without departing from the scope of the claims. In other words, unless a specific order of steps or actions is specified, the order and/or use of specific steps and/or actions may be modified without departing from the scope of the claims. Further, the various operations of methods described above may be performed by any suitable means capable of performing the corresponding functions. The means may include various hardware and/or software component(s) and/or module(s), including, but not limited to a circuit, an application specific integrated circuit (ASIC), or processor. Generally, where there are operations illustrated in figures, those operations may have corresponding counterpart means-plus-function components with similar numbering.

The following claims are not intended to be limited to the embodiments shown herein, but are to be accorded the full scope consistent with the language of the claims. Within a claim, reference to an element in the singular is not intended to mean “one and only one” unless specifically so stated, but rather “one or more.” Unless specifically stated otherwise, the term “some” refers to one or more. No claim element is to be construed under the provisions of 35 U.S.C. § 112(f) unless the element is expressly recited using the phrase “means for” or, in the case of a method claim, the element is recited using the phrase “step for.” All structural and functional equivalents to the elements of the various aspects described throughout this disclosure that are known or later come to be known to those of ordinary skill in the art are expressly incorporated herein by reference and are intended to be encompassed by the claims. Moreover, nothing disclosed herein is intended to be dedicated to the public regardless of whether such disclosure is explicitly recited in the claims.

What is claimed is:

1. A system comprising:
one or more input devices of a vehicle configured to capture driver health state data pertaining to a health state of the driver; and
one or more processors configured to:
compare the captured driver health state data to a threshold health state parameter;
disable movement of the vehicle based on the comparison of the driver health state data to the threshold health state parameter;
engage a recuperation mode in which one or more vehicle devices are configured to facilitate recuperation of the driver; and
disengage the recuperation mode and enable movement of the vehicle based on an additional comparison of the driver health state data to the threshold health state parameter.

2. The system of claim 1, wherein the one or more processors are configured to compare the driver health state data against a baseline health state condition stored in a memory module.

3. The system of claim 1, wherein the one or more processors are configured to predict a recuperation time by comparing the driver health state data against a health profile of the driver.

4. The system of claim 1, wherein the one or more vehicle devices comprise a user interface.

5. The system of claim 4, wherein, in the recuperation mode, the one or more processors are configured to provide an activity comprising at least one of games, puzzles, or tasks on the user interface.

6. The system of claim 5, wherein the one or more processors are configured to evaluate a performance metric of the activity to disengage the recuperation mode and enable movement of the vehicle.

7. The system of claim 1, wherein the one or more vehicle devices are one or more of lights with a selectively variable brightness, speakers configured to play audio, or aroma dispensers to facilitate recuperation of the driver.

8. A method comprising:
capturing driver health state data pertaining to a health state of a driver using one or more input devices positioned within a vehicle;
comparing the captured driver health state data using one or more processors to a threshold health state parameter;
disabling movement of the vehicle based on the comparison of the driver health state data to the threshold health state parameter;
engaging a recuperation mode, with the one or more processors, in which one or more vehicle devices are configured to facilitate recuperation of the driver based on the comparison of the driver health state data to the threshold health state parameter;
repeating capturing the driver health state data and comparing the captured driver health state data throughout a duration of the recuperation mode to monitor the health state of the driver; and
disengaging the recuperation mode and enabling movement of the vehicle based on an additional comparison of the driver health state data to the threshold health state parameter.

9. The method of claim 8, further comprising predicting a recuperation time to determine a duration of the recuperation mode.

10. The method of claim 8, further comprising determining a baseline health state condition and comparing the captured driver health state data against the baseline health state condition.

11. The method of claim 8, further comprising providing passive treatments including one or more of aroma therapy, calming audio, and calming visuals in the recuperation mode.

12. The method of claim 8, further comprising providing an activity including one or more of games, puzzles, and tasks on a user interface in the recuperation mode.

13. The method of claim 12, wherein disengaging the recuperation mode and enabling movement of the vehicle further comprises evaluating a performance metric of the activity.

14. The method of claim 8, wherein repeating capturing the driver health state data and comparing the captured driver health state data further comprises a plurality of predetermined check points.

15. A vehicle comprising:
one or more input devices configured to capture driver health state data pertaining to a health state of the driver;
one or more processors; and
a memory module storing instructions that, when executed by the one or more processors, cause the vehicle to perform operations comprising:
capturing driver health state data pertaining to a health state of a driver using the one or more input devices;
comparing the captured driver health state data to a threshold health state parameter;
disabling movement of the vehicle based on the comparison of the driver health state data to the threshold health state parameter;
engaging a recuperation mode in which one or more vehicle devices are configured to facilitate recuperation of the driver based on the comparison of the driver health state data to the threshold health state parameter;
repeating capturing the driver health state data and comparing the captured driver health state data throughout a duration of the recuperation mode to monitor the health state of the driver; and
disengaging the recuperation mode and enabling movement of the vehicle based on an additional comparison of the driver health state data to the threshold health state parameter.

16. The vehicle of claim 15, the operations further comprising predicting a recuperation time to determine a duration of the recuperation mode.

17. The vehicle of claim 15, the operations further comprising determining a baseline health state condition and comparing the captured driver health state data against the baseline health state condition.

18. The vehicle of claim 15, wherein the one or more vehicle devices are configured to facilitate recuperation of the driver by providing passive treatments including one or more of aroma therapy, calming audio, and calming visuals.

19. The vehicle of claim 15, wherein the one or more vehicle devices are configured to facilitate recuperation of the driver by providing an activity including one or more of games, puzzles, and tasks on a user interface.

20. The vehicle of claim 19, wherein disengaging the recuperation mode and enabling movement of the vehicle further comprises evaluating a performance metric of the activity.
